(19) United States
(12) Patent Application Publication
(10) Pub. No.: US 2025/0179041 A1
(43) Pub. Date: Jun. 5, 2025

(54) METHOD FOR THE PREPARATION OF ALKENE OXIDES USING OZONE AT ROOM TEMPERATURE

(71) Applicant: University of Saskatchewan, Saskatoon (CA)

(72) Inventors: Jafar Soltan, Saskatoon (CA); Nazanin Charchi Aghdam, Saskatoon (CA)

(73) Assignee: University of Saskatchewan, Saskatoon, SK (CA)

(21) Appl. No.: 18/840,688

(22) PCT Filed: Feb. 24, 2023
(86) PCT No.: PCT/CA2023/050234

§ 371 Date: Aug. 22, 2024

Related U.S. Application Data
(60) Provisional application No. 63/313,834, filed on Feb. 25, 2022.

(51) Int. Cl. C07D 301/10; B01J 21/04; B01J 21/06; B01J 23/50; B01J 35/30; B01J 35/61; B01J 37/02; B01J 37/08

(52) U.S. Cl. CPC C07D 301/10; B01J 21/04; B01J 21/063; B01J 23/50; B01J 35/394; B01J 35/612; B01J 35/613; B01J 35/615; B01J 37/0201; B01J 37/08

(57) ABSTRACT

The present application includes a method for preparing an alkylene oxide from an alkene that comprises reacting the alkene with ozone in the presence of a silver catalyst under conditions for selective partial oxidation of the alkene to provide the alkylene oxide at low temperatures.

### CROSS-REFERENCE TO RELATED APPLICATIONS

The present application claims the benefit of priority from U.S. provisional patent application No. 63/313,834 filed on Feb. 25, 2022, the contents of which are incorporated herein by reference in their entirety.

### FIELD

The present application relates generally to a method for the preparation of alkene oxides from alkenes at low temperature and, more particularly, to a method for the preparation of alkene oxides from alkenes by selective partial oxidation at, for example, room temperature.

### INTRODUCTION

Currently, ethylene oxide is produced by selective gas-phase partial oxidation of ethylene (ethylene epoxidation) with supported Ag/α-Al₂O₃ catalysts at a temperature range of 230-270° C. and pressure of 1-3 MPa [1]. An important parameter for the process is the activity and selectivity of the catalysts towards ethylene oxide production. Eq 1. represents the desired selective epoxidation of ethylene to produce ethylene oxide. However, this desired reaction is accompanied by two thermodynamically favored side reactions including total oxidation of ethylene (Eq. 2) and oxidation of ethylene oxide (Eq. 3). These side reactions produce CO₂ and water and are great challenges for reaching high selectivity to ethylene oxide.

C₂H₄+½O₂→C₂H₄O (1)

C₂H₄+3O₂→2CO₂+2H₂) (2)

C₂H₄O 5/2O₂→2CO₂+2H₂O (3)

Silver (Ag), due to its unique electronic properties, is considered one of the most active and selective catalysts for ethylene oxide production. Generally, 7-20 wt. % Ag is deposited on a porous support material. Promoters are introduced to the supported Ag/α-Al₂O₃ catalysts to enhance their selectivity to ethylene oxide. The advances made in silver-based catalysts since their original discovery have improved selectivity from 50% to about 90%. Ethylene oxide selectivity of around 90% can be achieved by the addition of solid promoters such as Cs, Re, and Mo to the catalyst. In addition, a gaseous chlorine promotor such as vinyl chloride is added to the reactor at ppm levels to increase the selectivity by modification of the active sites [4]. In the absence of the chlorine promoter, the rate and ethylene oxide selectivity decrease dramatically over time.

Partial oxidation of ethylene to produce ethylene oxide by silver catalysts has been a very active research topic because of its industrial importance and unsolved questions about the reaction mechanism and the nature of the active and selective sites. The nature of active and selective Ag species is still unresolved. Some studies suggest that the Ag surface needs to be oxidized to generate the catalytic active oxygen sites. These oxygen sites are considered to be responsible for ethylene oxidation [1]. Also, it has been reported that the silver phase remains metallic [5]. It is believed that there are several oxygen species on/in the silver surface, but their molecular structures have not been resolved. Electrophilic oxygen species are proposed to be responsible for ethylene epoxidation to ethylene oxide. The amount and distribution of oxygen species on/in the silver surface is influenced by the addition of promoters. Also, it has been suggested that promoters affect the electronic charge of the surface and subsurface Ag atoms, but the mechanism is still unknown [14, 15].

U.S. Pat. No. 2,769,016 discloses a method of catalytic oxidation of ethylene to ethylene oxide with a mixture of ozone and oxygen-containing gas such as air. The method occurs at a temperature of 200° C. to 400° C.

U.S. Pat. No. 6,765,101 discloses a method for synthesizing alkylene oxide with a source of oxygen, such as oxygen, ozone, and nitrogen oxides. The metal oxide catalyst can be supported on a carrier such as cerias, titanias, zirconias, silicas, and aluminas. No reaction using a silver catalyst and ozone at a low temperature is performed.

U.S. Pat. No. 6,348,607 discloses a method of oxidizing a C—C double bond-containing organic compound with a heterogenous catalyst in a reaction medium that comprises carbon monoxide. The oxygen source can be molecular oxygen, but can also be ozone or nitrogen oxides. This patent does not contain examples of ethylene oxidation.

U.S. Pat. No. 5,939,569 discloses a process for converting an olefin to the corresponding epoxide wherein the olefin, hydrogen, and oxygen are contacted with a catalyst comprising gold on a zirconium-containing support such as zirconia. No oxidations were actually performed using ozone.

### SUMMARY

The present application discloses a method for the preparation of alkene oxides from alkenes by selective partial ozone-based oxidation at low temperature, such as at room temperature. In the process of the present application, the alkylene oxides are produced by reacting alkene with ozone as the main reactant and in the presence of a supported silver catalyst. Catalytic ozonation of the present application can also be applied for other partial oxidation processes in the industry.

The present application therefore includes a method for preparing an alkylene oxide from an alkene comprising:
reacting the alkene with ozone in the presence of a silver catalyst under conditions for selective partial oxidation of the alkene to provide the alkylene oxide, wherein the conditions comprise a temperature of about 0° C. to about 500° C.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Certain embodiments of the application will now be described in greater detail with reference to the attached drawings in which:

FIG. 1 shows reaction rate and ethylene oxide yield versus ethylene partial pressure at different ozone partial pressures (600×10⁻⁶-1600×10⁻⁶ atm) and constant temperature a) 25° C., b) 50° C., and c) 75° C., in exemplary embodiments of the application.

FIG. 2 shows reaction rate and ethylene oxide yield versus ozone partial pressure at different ethylene partial pressures (40×10⁻⁶-120×10⁻⁶ atm) and constant temperature of a) 25° C., b) 50° C., and c) 75° C., in exemplary embodiments of the application.

FIG. 3 shows reaction rate and ethylene oxide yield versus reaction temperature at different ozone partial pressures (600×10⁻⁶-1600×10⁻⁶ atm) and constant ethylene partial pressure of a) 40×10⁻⁶ atm, b) 80×10⁶ atm, and c) 120×10⁻⁶ atm, in exemplary embodiments of the application.

FIG. 4 shows reaction rate and ethylene oxide yield versus reaction temperature at different ethylene partial pressure (40×10⁻⁶-120×10⁻⁶ atm) and constant ozone partial pressure of a) 600×10⁻⁶ atm, b) 1200×10⁻⁶ atm, and c) 1600×10⁻⁶ atm, in exemplary embodiments of the application.

FIG. 5 shows a summary of a) ethylene oxide yield, and b) reaction rate at different partial pressure of ethylene (40×10⁻⁶-120×10⁻⁶ atm) and ozone (600×10⁻⁶-1600×10⁻⁶ atm) versus reaction temperature, in exemplary embodiments of the application.

FIG. 6 shows reaction rate (upper line) and ethylene oxide yield (lower line) in the catalytic ozonation of ethylene over Ag (5%)/γ-Al₂O₃ at 25° C., in exemplary embodiments of the application.

FIG. 7 shows Ag K edge a) X-ray absorption near-edge structure (XANES) and b) Fourier-transformed X-ray absorption fine structure (FT-EXAFS) spectra in R space for AgOx (5%)/γ-Al₂O₃ catalyst fresh, spent in ethylene, O₃, under reaction condition (used), Ag₂O, and Ag foil (without phase shift correction), in exemplary embodiments of the application.

FIG. 8 shows TEM images of a) fresh and b) used Ag (5%)/γ-Al₂O₃, in exemplary embodiments of the application.

FIG. 9(a) and (b) show comparisons of reaction rate and product yield over low surface area Ag/α-Al₂O₃ and high surface area Ag/γ-Al₂O₃, in exemplary embodiments of the application.

### DETAILED DESCRIPTION

### Definitions

Unless otherwise indicated, the definitions and embodiments described in this and other sections are intended to be applicable to all embodiments and aspects of the present application herein described for which they are suitable as would be understood by a person skilled in the art.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “alkene” as used herein refers to any organic compound comprising a carbon-carbon double bond.

The term “selective partial oxidation” as used herein refers to a selective alkylene oxidation reaction, in which only alkylene oxide is produced.

The term “promoter” as used herein refers to a substance incorporated into the catalyst or mixed with the catalyst to improve its performance, for example, by increasing catalyst activity or selectivity.

The term “inhibitor” as used herein refers to a compound that is added to the gas feed to improve ethylene oxide selectivity, for example, by suppressing the complete oxidation of ethylene to carbon dioxide and water [18].

The term “silver catalyst of the present application” as used herein refers to the silver catalyst as described herein.

The term “method of the present application” as used herein refers to the method for preparing an alkylene oxide from an alkene as described herein.

### Methods of the Application

Decomposition of ozone, according to the present application, may follow the following reaction pathway: (* denotes the surface site on the catalyst):

O₃+*→O₂+O* (4)

O*+O₃→O₂+O₂* (5)

O₂*→O₂+* (6)

where ozone gas (O₃) is converted to oxygen (O₂) and active oxygen species (O*). It has been shown that atomic oxygen species (O₂* and O*) are responsible for oxidation in the presence of ozone. Molecular oxygen species are spectators and do not contribute to the oxidation reaction. These are generated due to catalytic self-decomposition of ozone as indicated in Eq. (4) [12,13].

However, catalytic oxidation of ethylene to produce ethylene oxide does not take place at room temperature in the absence of ozone. Thus, the catalytic ozonation of ethylene for the ethylene oxide process of the present application is a unique process in that ozone is the main reactant and not a promoter.

Accordingly, the present application includes a method for preparing an alkylene oxide from an alkene comprising reacting the alkene with ozone in the presence of a silver catalyst under conditions for selective partial oxidation of the alkene to provide the alkylene oxide, wherein the conditions comprise a temperature of about 0° C. to about 500° C.

In some embodiments, the temperature is about 0° C. to about 300° C., about 0° C. to about 150° C., about 10° C. to about 100° C., about 15° C. to about 50° C., about 20° C. to about 30° C., or about 25° C. In some embodiments, the temperature is about 25° C.

The alkenes which can be used in the method of the present application are any alkenes know in the art. This includes linear, branched or cyclic hydrocarbons comprising at least one double bond. The hydrocarbon can be a compound containing only carbon and hydrogen atoms, but can also be substituted, for example, with one or more halo, ester and/or alcohol moieties and/or the like.

Examples of suitable alkenes include, but are not limited to, ethylene, propylene, 1-butene, 2-butene, isobutylene, butadiene, 1-pentene, 2-pentene, isoprene, 1-hexene, 3-hexene, 1-heptene, 1-octene, diisobutylene, 1-nonene, 1-decene, 1-undecene, 1-dodecene, 1-tridecene, 1-tetradecene, 1-pentadecene, 1-hexadecene, 1-heptadecene, 1-octadecene, 1-nonadecene, 1-eicosene, polybutadiene, polyisoprene, cyclopentene, cyclohexene, cycloheptene, cyclooctene, cyclooctadiene, cyclododecene, allyl chloride, allyl bromide, acrylic acid, methacrylic acid, crotonic acid, vinylacetic acid, crotyl chloride, methallyl chloride, dichlorobutenes, allyl alcohol, allyl carbonate, allyl acetate, alkyl acrylates (e.g. methacrylates), diallyl maleate, diallyl phthalate and the like.

In some embodiments, the alkene is selected from ethylene, propylene, 1-butene, 2-butene, isobutylene, butadiene, pentenes, isoprene, 1-hexene, 3-hexene, 1-heptene, 1-octene, diisobutylene, 1-nonene and 1-decene.

In some embodiments, the alkene is ethylene and the alkylene oxide is ethylene oxide.

Silver has an oxidation state of between −2 and +3. The oxidation state of silver varies based on the preparation method, metal loading and/or type of support used for preparing the silver catalyst of the present application. In some embodiments, the silver used in the method of the present application has an oxidation state of 0, +1 or a combination thereof. Thus, the silver catalyst used in the method of present application may be pure silver or a silver compound. Examples of suitable silver compounds include, but are not limited to, silver oxide, silver acetate, silver sulfate, silver bromide, silver phosphate, silver fluoride, silver nitrate and the like.

In some embodiments, the silver catalyst is silver oxide.

In some embodiments, the silver catalyst is on a support. The silver may be deposited chemically or mechanically from suspensions of finely divided silver or silver oxide on the support. Examples of suitable techniques for the deposition of silver catalysts include, but are not limited to, impregnation, co-precipitation, chemical vapor deposition, ion-exchange, and deposition by precipitation. The silver catalyst is loaded on the support in an amount sufficient to allow the desired level of activity of the catalyst.

In some embodiments, the support is Al₂O₃ or a semi-conducting metal oxide.

In some embodiments, the semi-conducting metal oxide is TiO₂, ZnO or CeO₂.

In some embodiments, the support is γ-Al₂O₃. In some embodiments, the support is α-Al₂O₃.

In some embodiments, the γ-Al₂O₃ has a surface area of about 50 m²/g to about 500 m²/g, about 80 m²/g to about 400 m²/g, about 100 m²/g to about 350 m²/g, about 150 m²/g to about 300 m²/g, or about 200 m²/g to about 240 m²/g. In some embodiments, the γ-Al₂O₃ support has a surface area of about 200 m²/g to about 240 m²/g.

In some embodiments, the α-Al₂O₃ has a surface area of about 2 m²/g to about 15 m²/g, about 4 m²/g to about 10 m²/g, or about 6 m²/g. In some embodiments, the α-Al₂O₃ has a surface area of about 6 m²/g.

TiO₂ exists in three polymorphs, namely, anatase, rutile, and brookite. In some embodiments, the TiO₂ used in the method of the present application comprises an anatase form or a rutile form or a mixture thereof. In some embodiments, the TiO₂ used in the method of the present application is pure anatase or pure rutile crystalline forms.

In some embodiments, the TiO₂ is a rutile crystalline form. In some embodiments, the TiO₂ is an anatase crystalline form.

In some embodiments, the TiO₂ used in the method of the present application has an average particle size of about 2 nm to about 800 nm.

In some embodiments, when the rutile form of TiO₂ is used, the average particle size is of about 300 nm to about 700 nm, about 400 nm to about 600 nm, or about 500 nm. In some embodiments, the average particle size of TiO₂ rutile is about 500 nm.

In some embodiments, when the anatase form of TiO₂ is used, the average particle size is about 3 to about 10 nm, or about 5 nm. In some embodiments, the average particle size of TiO₂ anatase is about 5 nm.

In some embodiments, the TiO₂ has a surface area of about 5 m²/g to about 500 m²/g.

In some embodiments, when the anatase from of TiO₂ is used, the TiO₂ anatase has a surface area of about 50 m²/g to about 450 m²/g, or about 100 m²/g to about 430 m²/g. In some embodiments, the TiO₂ anatase has a surface area of about 420 m²/g.

In some embodiments, when the rutile form of TiO₂ is used, the TiO₂ rutile has a surface area of about 7 m²/g to about 30 m²/g, or about 10 m²/g to about 20 m²/g. In some embodiments, the TiO₂ rutile has a surface area of 15 m²/g.

In some embodiments, the ZnO or CeO₂ have a surface area of about 7 m²/g to about 25 m²/g. In some embodiments, the ZnO has a surface area of about 25 m²/g. In some embodiments, the CeO₂ has a surface area of about 8 m²/g.

In some embodiments, the ZnO used in the method of the present application has an average particle size of about 5 nm to about 30 nm, about 10 nm to about 20 nm, or about 18 nm. In some embodiments, the average particle size of ZnO is about 18 nm.

The particle size represents an average size of the particles and can be measured by any known method in the art, such as dynamic image analysis (DIA), static laser light scattering (SLS) or dynamic light scattering (DLS).

In some embodiments, the silver catalyst is loaded on the support in an amount of about 0.1 wt % to about 30 wt %, about 1 wt % to about 20 wt %, about 3 wt % to about 15 wt %, or about 5 wt % to about 10 wt % based on the total weight of the catalyst. In some embodiments, the silver catalyst is loaded on the support in an amount of about 5 wt %. In some embodiments, the silver catalyst is loaded on the support in an amount of about 10 wt %.

In some embodiments, the silver catalyst is 10 wt % AgOₓ supported on γ-Al₂O₃ wherein x varies depending on the oxidation state of Ag as described above. In some embodiments, the γ-Al₂O₃ has a surface area of about 219 m²/g.

In some embodiments, the silver catalyst is 10 wt % AgOₓ supported on TiO₂ anatase, wherein x varies depending on the oxidation state of Ag as described above. In some embodiments, the TiO₂ anatase has a surface area of about 420 m²/g.

In some embodiments, the conditions for selective partial oxidation of the alkene to provide the alkylene oxide further comprise a partial pressure of the alkene of about 10×10⁻⁶ atm to about 0.5 atm.

In some embodiments, the conditions for selective partial oxidation of the alkene to provide the alkylene oxide further comprise a partial pressure of the ozone of about 10×10⁻⁶ atm to about 0.8 atm.

In some embodiments, the concentration of the ozone is higher than that of the alkene to allow enhanced conversion and selectivity of the alkenes to the alkylene oxides according to the method of the present invention.

Monoatomic oxygen adsorption can result in the formation of alkylene oxide, as well as CO₂, and H₂O. One factor in determining the outcome is the charge state of the monoatomic oxygen. If the oxygen is strongly negatively charged, it behaves like a base and removes hydrogen from the alkylene, leading to complete combustion. However, if there is subsurface oxygen present, it competes with the adsorbed oxygen for silver electrons, which reduces the negative charge on the adsorbed oxygen. This makes it more likely to react with the electron-rich double bond of the alkylene and leads to the formation of alkylene oxide. In this case, the molecular structure of the alkylene remains intact and the path to forming alkylene oxide becomes dominant. In some embodiments, the silver catalyst of the application decomposes ozone to mainly electrophilic oxygen species which are electron deficient (with a less negative charge) which will be inserted into the C—C bond and produce alkylene oxide.

In some embodiments, the silver catalyst after the partial oxidation comprises metallic silver.

In some embodiments, the silver catalyst is free from a promoter. Thus, the silver catalyst can be synthesized and used for catalytic ozonation of the alkenes in the method of the present invention without promoters.

In some embodiments, one or more promoters and/or inhibitors are incorporated into the silver catalyst to improve its performance. Examples of such promoters are alkali metals selected from Group I of the Periodic Table such as lithium, sodium, potassium, rubidium and/or cesium, alkaline earth metals selected from Group Il of the Periodic Table such as beryllium, magnesium, calcium, strontium and/or barium, the lanthanide rare earth metals or the actinide metals. Examples of such inhibitors are chlorine compounds such as 1,2-dichloroethane, vinyl chloride, ethyl chloride and the like. In some embodiments, the amount of promoter and/or inhibitor is between about 0.01 and about 10 weight percent based on the total weight of the catalyst. In some embodiments, the addition of promoters and/or inhibitors improves the selectivity and the ethylene oxide yield in catalytic ozonation process of the present application.

In some embodiments, when the silver catalyst is supported on ZnO, CeO₂ or TiO₂ rutile, one or more promoters are added. In some embodiments, the promoter added to the low surface area support, such as α-Al₂O₃, ZnO, CeO₂ or TiO₂ rutile improves the selectivity and the ethylene oxide yield.

In some embodiments, the silver catalyst is diluted with helium. The diluent may be used to remove and dissipate the heat generated during the process of catalytic ozonation. Any gas which does not interfere with the catalytic ozonation reaction may be utilized, suitably one that is essentially inert (non-reactive) under the reaction conditions or any other gas that has high heat adsorption capacity. Suitable gaseous diluents, which are used when the reactants are in the vapor phase when contacted with the catalyst, include helium, nitrogen, argon, methane, ethane, propane, steam, carbon dioxide and the like and mixtures thereof. In some embodiments, the gaseous diluent is methane or carbon dioxide.

In some embodiments, the catalytic ozonation process of the application is carried out in a reactor of any conventional design suitable for vapor phase processes including, for example, batch, fixed bed, transport bed, fluidized bed, moving bed, shell tube, bubble column and trickle bed reactors. In some embodiments, the reactor is operated with continuous, intermittent, or swing flow. In some embodiments, the catalytic ozonation process of the present application is carried out as a gas phase process, which is a process wherein gaseous reactants are reacted under the influence of a solid catalyst.

In some embodiments, the catalytic ozonation process of the application is carried out in a packed bed reactor. In some embodiments, the reactor is made of a tube and a catalytic bed. In some embodiments, the reactor contains one or more tubes. The diameter of the tube can vary and is within the knowledge of a person skilled in the art. In some embodiments, the diameter of the tube is between about 5 mm to about 1 cm, more than about 1 cm, or more than about 1 meter. The length of the catalytic bed can vary depending on the material of the catalyst support. In some embodiments, the length of the catalytic bed is between about 5 mm to about 1 cm, more than 1 cm, or more than about 1 meter.

In some embodiments, oxygen stream is passed through an ozone generator to produce ozone. In some embodiments, the ozone is mixed with the stream of alkylene and diluent and passes through the catalytic bed of the reactor. In some embodiments, the catalytic bed comprises the silver catalyst of the application loaded on the support as described in the application.

In some embodiments, the total flow rate of the gas mixture at the inlet of the reactor is about 500 sccm (standard cubic centimeters per minute) to about 1500 sscm, or about 1,000 sccm. In some embodiments, the weight hourly space velocity in the reactor (WHSV) is about 100 lh-1 g-1 to about 500 lh-1 g-1, about 200 lh-1 g-1 to about 400 lh-1 g-1, or about 300 lh-1 g-1.

In some embodiments, the amount of the catalyst varies depending, for example, on the amount of the alkylene and the size of the reactor and is within the knowledge of a person skilled in the art. For example, in some embodiments, for a reactor that is a tube having an internal diameter of 0.5 inches and 0.12 sccm of alkylene, about 0.1 g to about 0.3 g, or about 0.2 g of catalyst is used.

In some embodiments, the concentration ratio of alkylene to ozone is from about 1:5 to about 1:15, about 1:7 to about 1:13, or about 1:9 to about 1:12. In some embodiments, the concentration ratio of alkylene to ozone is about 1:10.

The following non-limiting examples are illustrative of the present application:

### EXAMPLES

### General Methods and Materials

All chemicals are commercially available and used as received without any further purification. Silver nitrate (AgNO₃, 99.9%), γ-Al₂O₃ (S_{BET}=219 m²/g), and α-Al₂O₃ (surface area of 6 m²/g) were purchased from Alfa Aesar. TiO₂ anatase (5 nm, S_{BET}=420 m²/g), TiO₂ rutile (500 nm, S_{BET}=15 m²/g), and ZnO (18 nm, S_{BET}=25 m²/g) were purchased from US research nanomaterial. CeO₂ (S_{BET}=8 m²/g) was purchased from Sigma-Aldrich.

### Preparation and Characterization of the Catalyst

Supported AgOₓ catalysts were prepared by dry impregnation of different supports using silver nitrate as a precursor. In a typical synthesis procedure, a certain amount of silver nitrate (nominal mass of 1-20 wt. % Ag per mass of the catalyst) was dissolved in distilled water and mixed over a stirrer for 1 hour. Then the solution was added dropwise to the support under vigorous stirring to form a uniform paste. Finally, the resulting paste (impregnated support) was dried in an oven at 80° C. for 10 hours and then calcined in air at 350° C. for 2 hours. Alternatively, the metal salt loaded support may be heated to 100-900° C. for 1 to 5 hours.

Brunauer-Emmett-Teller (BET) surface area of the supports was determined by N₂ adsorption using ASAP™ 2020 (Micromeritics) instrument. X-ray absorption spectroscopy (XAS) analysis of the fresh and spent catalysts was performed at the hard X-ray micro analysis (HXMA) beamline of the Canadian Light Source. Data processing and linear combination fitting (LCF) of Ag K-edge X-ray absorption near-edge structure (XANES) data were performed by Athena software. R-space curve fittings for X-ray absorption fine structure (EXAFS) data were conducted using the software WinXas 3.240 based on theoretical phases and amplitude functions generated from FEFF7.

### Catalyst Activity Measurements

For evaluation of the catalysts' activity, oxygen stream passed through an ozone generator (AZCO Industries LTD, HTU-500S) to produce ozone. The gas mixture of ozone, oxygen, and inert gas (i.e. nitrogen, helium) passed through the catalytic beds (AgOₓ on Al₂O₃, TiO₂, CeO₂, or similar semiconductors). The reactor operating at room temperature was made of a single tube (i.d. ½ in.) installed inside an oven (Binder™, FP 115) which controlled the reaction temperature. The total flow rate at the inlet of the reactor was 1,000 ml/min resulting in the weight hourly space velocity (WHSV) of 300 hr⁻¹/g⁻¹. The ozone/oxygen, ethylene, and inert gas streams were combined to produce the reactor feed mixture. The concentration of ozone introduced into the reaction zone was higher than the concentration of ethylene. The amount of ozone depends on variety of operation conditions e.g., ethylene concentration, total flow rate, type, and amount of catalyst and can be routinely determined by one skilled in the art. An inlet ozone/ethylene concentration ratio of 10 has been used as an example.

The exhaust stream of the reactor passed through a long-path gas cell (PIKE, volume 0.1 L, 2.4 m optical length, KBr window), coupled with a Fourier transform infrared spectroscopy (FTIR) spectrometer (Nicolet™ iS50). Spectra were collected at a resolution of 4 cm⁻¹ in the range of 4000-400 cm⁻¹. An ozone monitor (Teledyne API M454) was employed to measure ozone content in the exhaust stream.

Ethylene conversion, ethylene oxide yield, and ozone conversion were calculated using the following equations:

$$Et conversion = 100 \times \frac{{[ E t ]}_{i n} - {[ E t ]}_{o u t}}{{[ E t ]}_{i n}} \qquad ( 7 )$$

$$EO yield = 100 \times \frac{[ E O ]}{{[ E t ]}_{R e a c t e d}} \qquad ( 8 )$$

$${O}_{3} conversion = 100 \times \frac{{[ {O}_{3} ]}_{i n} - {[ {O}_{3} ]}_{out}}{{[ {O}_{3} ]}_{i n}} \qquad ( 9 )$$

### Kinetic Data Collection

Kinetic study experiments were performed over AgOₓ/γ-Al₂O₃ with silver loading of 5 wt. %. The inlet gas was fed into a tubular reactor loaded with approximately 20 mg of the catalyst. The catalyst was diluted with silica carbide to prevent gas phase channeling and temperature gradients along the catalyst bed. The total flow rate was set at 2000 STP mL min⁻¹ for all experiments with different ethylene and ozone concentrations. The employed catalyst powder size, weight, reaction conditions, and reactor configurations removed mass transfer limitations, resulting in kinetically controlled operating conditions.

### Kinetic Data for Catalytic Reaction of Ozonation of Ethylene

A kinetic model was developed to describe the kinetic data obtained from catalytic ozonation of ethylene. The reaction was carried out in an atmospheric pressure, continuous plug flow reactor. Kinetic data was obtained at three different temperature levels (25, 50, and 75° C.) and atmospheric pressure. Catalytic ozonation of ethylene over AgOₓ (5%)/γ-Al₂O₃ was performed with either constant ethylene partial pressure while ozone partial pressure was changed between 600 and 1600×10⁻⁶ atm, or constant ozone partial pressure with ethylene partial pressure of 40 to 120×10⁻⁶ atm.

The power law model for catalytic ozonation of ethylene is expressed by

$$- {r}_{e t h} = k {C}_{e t h}^{n} {C}_{{O}_{3}}^{m} \qquad ( 10 )$$

where
rₑₜₕ: ethylene reaction rate (mole kg_{cat}⁻¹ s^{31 1})
Cₑₜₕ: concentration of ethylene (mole L⁻¹)
C_{O3}: concentration of ozone (mole L⁻¹).

The power law model consists of three unknown parameters, as stated in Eq. 10. In this model, k is the reaction rate constant, while n and m express the dependency of the reaction rate on the concentration of ethylene and ozone, respectively.

Reaction rate data were fitted to Eq. 10 by using nonlinear least-squares regression analysis. After the kinetic parameters were calculated for each temperature, Arrhenius equation (Eq. 11) was employed to determine the pre-exponential factor k₀ and apparent activation energy Eₐ. R is the universal gas constant (8.314 J K⁻¹ mol⁻¹) and T is the reaction temperature in Kelvin.

$$L n k = {Lnk}_{°} + ( \frac{- {E}_{a}}{R} ) \frac{1}{T} \qquad ( 11 )$$

The results of the power late rate modeling are summarized in Table 1.

TABLE 1
Fitting results for power law kinetic model
$Power law rate expression : {r}_{ethylene} = {kC}_{ethylene}^{n} {C}_{{O}_{3}}^{m} , k = {K}_{0} exp ( \frac{- {E}_{a}}{RT} )$

| n | m | k₀(mol^{0.15} L^{0.85} kg⁻¹ s⁻¹) | Eₐ (kJ mol⁻¹) | R² * |
|---|---|---|---|---|
| 0.33 | 0.52 | 64.83 | 22.1 | 0.9987 |

* Coefficient of determination of the regression analysis.

The reaction orders with respect to ethylene and ozone are 0.33 and 0.52, respectively. These positive values for reaction order demonstrate that an increase in the concentration of ethylene or ozone enhances the reaction rate. However, the concentration of ozone has a greater impact on the reaction rate compared to ethylene. The apparent activation energy (Eₐ) of the catalytic ozonation reaction is determined to be 22.1 KJ mol⁻¹. Activation energies in the range of 45-90 KJ mol⁻¹ have been reported for the epoxidation (ethylene oxide production) and 42-122 kJ mol⁻¹ for the total oxidation (CO₂ production) [2,3]. However, the most commonly reported activation energy is in the range of 90-100 KJ mol⁻¹ [1].

While not wishing to be limited by theory, the significant decrease of the activation energy may allow effective oxidation of ethylene by ozone (catalytic ozonation), thus making oxidation of ethylene at room temperature possible.

### Operating Parameters

The possible general reactions of ozone and ethylene can be expressed by the following reactions:

C₂H₄+⅓O₃→C₂H₄O (12)

C₂H₄+2O₃→2CO₂+2H₂O (13)

C₂H₄O+ 5/3O₃→2CO₂+2H₂O (14)

The effect of operating conditions including reactants' partial pressures and reaction temperature on the reaction rate and ethylene oxide yield was investigated. No CO was detected in catalytic ozonation of ethylene over AgOₓ (5%)/γ-Al₂O₃ and ethylene oxide and CO₂ were the main reaction products.

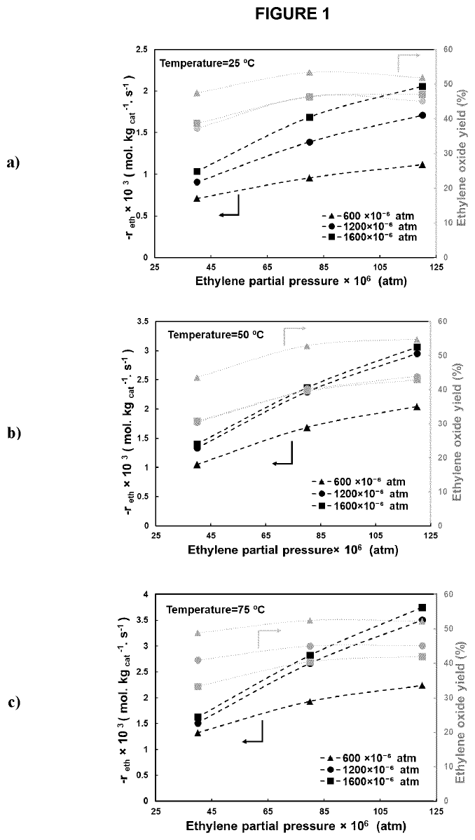

The effect of ethylene partial pressures on ethylene reaction rate and ethylene oxide yield at different ozone partial pressures and constant temperatures is shown in FIG. 1a-c. At all the temperatures, at a constant ozone partial pressure, the reaction rate increases with an increase of ethylene partial pressure. This results in positive reaction order of ethylene as is reported in Table 1. However, a slight increase in ethylene oxide yield is observed by an increase in ethylene partial pressure, particularly at room temperature. For example, at room temperature (25° C.) and fixed ozone partial pressure of 1600×10⁻⁶ atm, the reaction rate increases from 0.9×10⁻³ mol·kg_{cat}⁻¹.s⁻¹ to 1.7×10⁻³ mol·kg_{cat}⁻¹.s⁻¹ by increasing ethylene partial pressure in the range of 40-120×10⁻⁶ atm, while ethylene oxide yield increases from 47% to 52%. It can be concluded that an increase in the partial pressure of ethylene favors the total oxidation of ethylene (Eq 10) and higher CO₂ production. Also, at all the temperatures, the highest reaction rate is obtained at the highest ozone partial pressure (1600×10⁻⁶ atm), while the highest ethylene oxide yield is achieved at the lowest ozone partial pressure (600×10⁻⁶ atm). It can be concluded that although an increase in ethylene partial pressure increases the reaction rate, it favors CO₂ production as well.

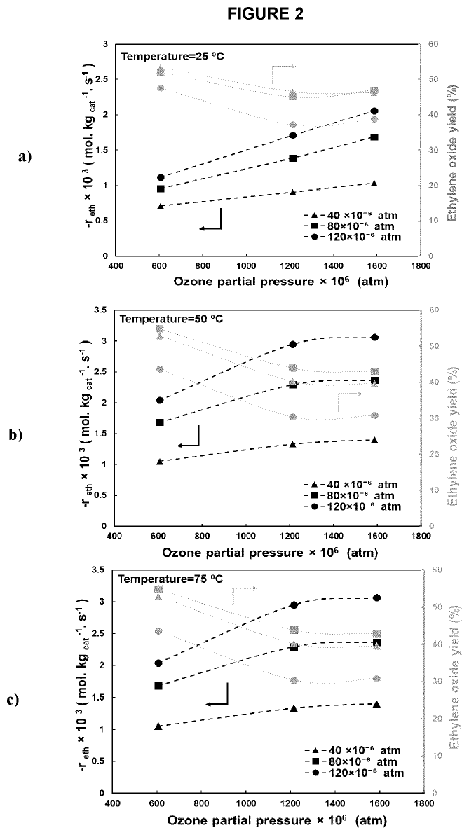

FIG. 2 a-c shows the effect of ozone partial pressure on reaction rate and ethylene oxide yield at different temperatures when ethylene partial pressure was kept constant. Similar to the results of the fixed partial pressure of ethylene, the reaction rate increases with increase of ozone partial pressure, indicating the positive reaction order with respect to ozone. On the other hand, a significant decrease in ethylene oxide yield is observed by increasing ozone partial pressure which indicates the increase in CO₂ production by increasing the ozone concentration and reaction rate (Eq 10 and 11). It can be concluded that lower conversions will benefit ethylene oxide production.

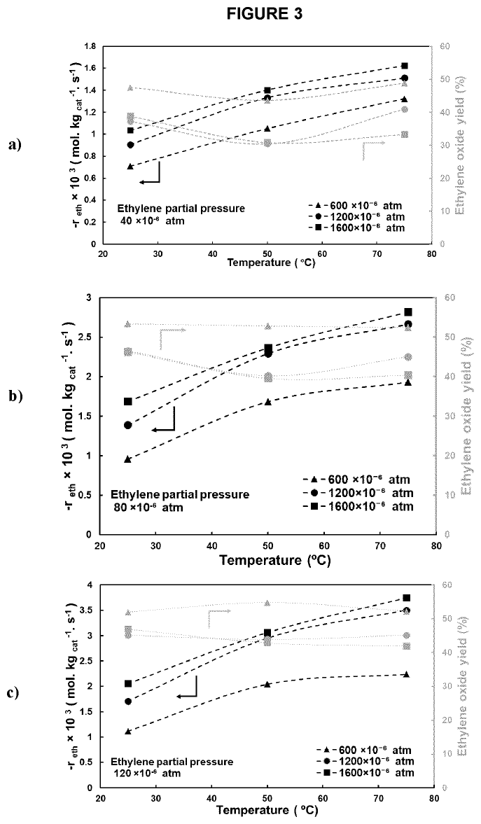

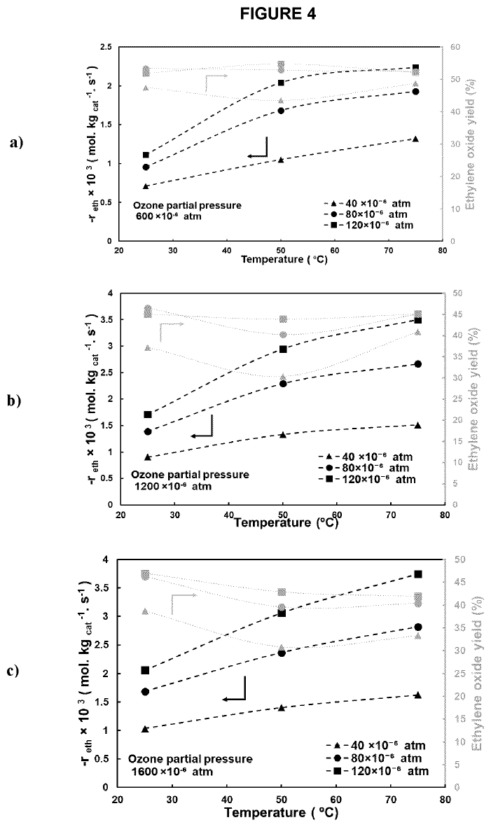

The reaction rates and ethylene oxide yield versus temperature at fixed ozone or ethylene partial pressures are shown in FIG. 3 a-c and FIG. 4 a-c. Generally, it can be observed that an increase in reaction temperature significantly enhances the reaction rate, but ethylene oxide yield stays unchanged or decreases.

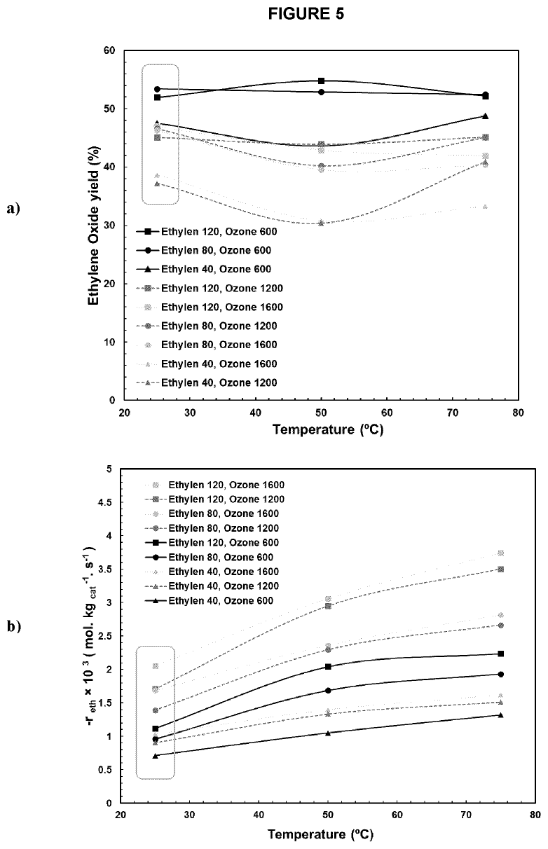

FIG. 5 a-b represents a summary of variation in reaction rate and ethylene oxide yield at different ethylene and ozone concentrations and temperatures. It can be seen that despite a low reaction rate, a high yield of ethylene oxide can be achieved at room temperature. An increase in reaction temperature increases the reaction rate but favors undesirable total oxidation of ethylene. According to this data, conducting the reaction at room temperature is likely to be more selective and desirable than at higher temperatures. Furthermore, room temperature catalytic ozonation of ethylene may save in energy and reduce carbon footprint.

### Catalytic Ozonation of Ethylene

Catalysts synthesized and used for catalytic ozonation of the ethylene were prepared without promotors. Also, no promotor was added to the reaction system. In the process of catalytic ozonation of ethylene of the present application, ozone is not a promotor. Ozone in this reaction is the main reactant and its concentration was higher than ethylene concentration to, for example, overcome the activation energy at room temperature. Ozone concentration is one of the parameters affecting the conversion and selectivity of the reaction as was observed above.

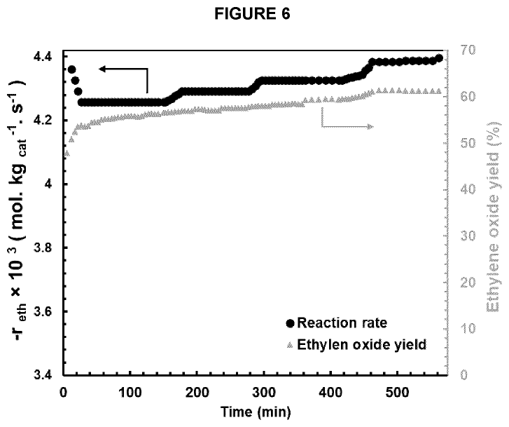

In industrial reactions for the production of ethylene, in the absence of promoters, a decrease in reaction rate and ethylene oxide selectivity occurs [1]. But in the present catalytic ozonation of ethylene over Ag(5%)/γ-Al₂O₃, in prolonged reaction time, an increase in reaction rate and ethylene oxide yields was observed (FIG. 6). This observation is contrary to what was observed for industrial ethylene oxide production reaction (without ozone) with no promotors added.

### Characterization of Catalysts

X-ray absorption fine structure spectroscopy (XAFS) was used to thoroughly characterize the atomic environment of the fresh and used catalysts. XAFS is a versatile tool to gain structural information because it can be used to determine the local environment around the selected atomic species, irrespective of the crystallinity or dimensionality of the target materials [16]. The X-ray absorption spectrum consists of two regimes including X-ray absorption near-edge structure (XANES) and extended X-ray absorption fine structure (EXAFS). XANES is especially sensitive to the formal oxidation state and coordination chemistry of the absorbing atom. Because of the sensitivity of the position and shape of the edge to the formal valence state and coordination environment, XANES can be used as a fingerprint to identify different phases. EXAFS measurements are sensitive to the bonding environment of the absorbing atom, and they are utilized to further study the atomic environment to determine the coordination number, atomic distances, and species of the neighboring atoms [17].

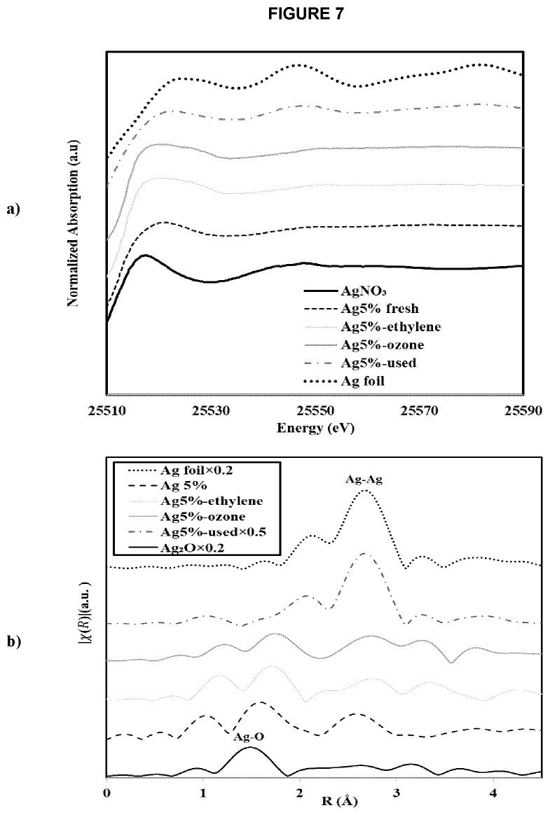

XANES and EXAFS analysis were performed on the Ag (5%)/γ-Al₂O₃ catalyst before (fresh) and after catalytic ozonation of ethylene (used). Ex-situ XANES (FIG. 7 a) and EXAFS (FIG. 7 b) spectra of the fresh and used catalysts showed a significant spectral evolution for Ag from Ag¹⁺ to metallic form. Based on the results of linear combination fitting, the abundance of the metallic Ag increased from 20% in fresh catalyst to 44% in the used one (Table 2). Moreover, in the fresh catalysts, 0.6Ag atoms were found at the second shell at the interatomic distance of 2.81 Å. However, in the case of used Ag 5%, 3.9 Ag atoms were found at the second shell at 2.85 Å (Table 3). While not wishing to be limited by theory, this may be attributed to the Ag₂O surface, which is not stable under the reaction conditions, and it may transform into a metallic nature by losing the weakly bound surface oxygen.

TABLE 2
Result of linear combination fitting of Ag K-edge XANES.

| Catalyst | Ag⁰ | Ag¹⁺ |
|---|---|---|
| AgOₓ (5%)/Al₂O₃ | 20 | 80 |
| AgOₓ (5%)/Al₂O₃-ethylene | 19 | 81 |
| AgOₓ (5%)/Al₂O₃—O₃ | 18 | 82 |
| AgOₓ (5%)/Al₂O₃-used | 44 | 56 |

TABLE 3
EXAFS fitting results for the fresh and spent Ag (5%)/ γ-Al₂O₃.

| Catalyst | Path | CN | R (Å) | σ²(Å²) |
|---|---|---|---|---|
| AgOₓ (5%)/Al₂O₃-fresh | Ag—O¹ | 0.6 | 2.20 | 0.0050 |
| | Ag—Ag² | 0.6 | 2.81 | 0.00950 |
| AgOₓ (5%)/Al₂O₃-ethylene | Ag—O | 0.5 | 2.23 | 0.0050 |
| | Ag—Ag | 0.6 | 2.79 | 0.00950 |
| AgOₓ (5%)/Al₂O₃—O₃ | Ag—O | 0.5 | 2.28 | 0.0050 |
| | Ag—Ag | 0.6 | 2.79 | 0.00950 |
| AgOₓ (5%)/Al₂O₃-under | Ag—O | 0.5 | 2.29 | 0.0050 |
| reaction | Ag—Ag | 3.9 | 2.85 | 0.00950 |

¹Ag—O of Ag₂O type (R = 2.04 Å)
²Ag—Ag of metallic type (R = 2.89 Å)

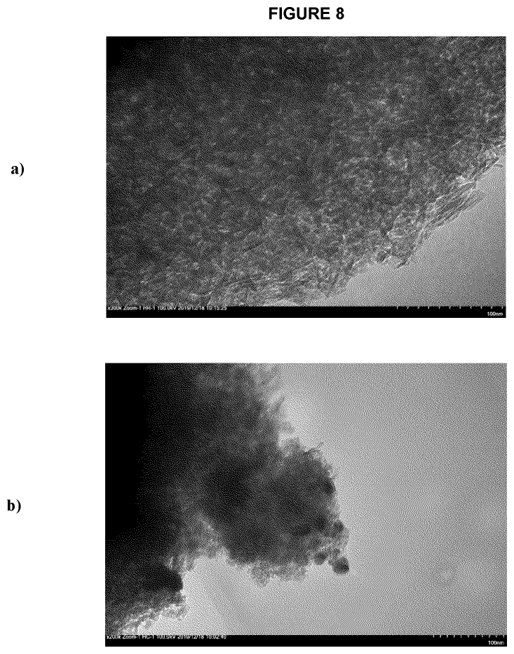

These results demonstrate that Ag oxidation state and atomic environment evolves dynamically under the reaction conditions. Comparing TEM images of the fresh and used catalysts (FIG. 8 a-b) showed that Ag particle size increased during the reaction which further confirms the reduction under the reaction conditions.

Changes in particle size and morphology have been reported for oxidation of ethylene (with no ozone) under industrial operating conditions and are suggested to be responsible for the decrease in rate and ethylene oxide selectivity. This change in Ag structure is considered to be undesirable in ethylene oxidation because enlarging Ag particles could lead to agglomeration and uneven distribution of the Ag particles [6]. On the other hand, it has been reported that Ag particle size (initial particle size before reaction) significantly affects the ethylene oxide selectivity; and larger particle sizes yield higher ethylene oxide selectivity [1].

It can be seen in FIG. 6, that the structural changes of Ag under reaction conditions, enhance the ozonation reaction rate and ethylene oxide yield. This is different from the oxidation of ethylene process. Moreover, the change in the structure of Ag in catalytic ozonation occurs at room temperature, whereas typically reduction of Ag occurs at higher temperatures.

To study the mechanism of reduction of Ag in catalytic ozonation of ethylene, the same Ag (5%)/γ-Al₂O₃ catalyst, was used with all the gases involved in the reaction (with the same concentration as in the reaction) including ethylene, ozone, CO/CO₂, and ethylene oxide, separately to study their role independently. No color change (a visual indication of the change in oxidation state) was observed for the spent catalyst in CO/CO₂, and ethylene oxide; so XANES and EXAFS analysis were not performed for these catalysts. Results of ex-situ XANES and EXAFS of the catalysts spent in CO/CO₂ and ethylene oxide are presented in FIG. 7 a-b, Table 2 and Table 3. No significant changes were found in the silver atomic structure of the Ag species that were exposed to ethylene and ozone. It is therefore concluded that the Ag reduction occurs under reaction conditions and in the presence of ethylene and ozone simultaneously. While not wishing to be limited by theory, this can be attributed to strong metal-support interaction (SMSI) under reaction conditions and electron transfer from support to the metal. It has been reported that interfacial contact between a metal and support can result in charge redistribution at the interface [7]. The strong interaction induces significant changes in electronic properties. This redistribution of electrons can change the oxidation state of the metal atoms of both active metal and support. Furthermore, this facilitates the adsorption, dissociation, and desorption of oxygen species [8-10]. It was shown that none of the gases involved in the reaction including ethylene, ozone, CO/CO₂, and ethylene oxide could severely affect the atomic structure of the Ag species in Ag (5%)/γ-Al₂O₃ at room temperature alone. While not wishing to be limited by theory, the presence of oxidizing agent (ozone) and reducing agents (ethylene, CO/CO₂, and ethylene oxide) may create a charge gradient that accelerates charge transfer from support to Ag particles at room temperature. This charge transfer may reduce Ag species over Al₂O₃ during the catalytic ozonation of ethylene.

Resonance structures of ozone are represented as follows:

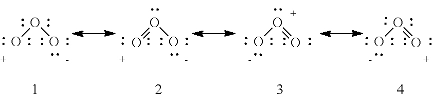

Electrophilic properties of ozone have been explained by using forms 1 and 4 of ozone, which have a positively charged terminal oxygen with only six electrons [11].

While not wishing to be limited by theory, an increase in the activity and selectivity of the Ag (5%)/γ-Al₂O₃ catalyst, may be attributed to the reduction of Ag particles under reaction condition and the increase in the concentration of electrophilic oxygen species on the surface of the catalyst (generated from ozone decomposition). The ozone may act as an electrophilic center which is an active site for ethylene oxidation. Also, the reduction of Ag during the reaction, may reconstruct its structure, and create more active sites for ethylene oxide production.

The double bond in ozone has a higher electron density than the single bond, making the central oxygen atom more electron-deficient and therefore more electrophilic. As an electrophile, ozone can react with nucleophiles, which are electron-rich species that have lone pairs of electrons or IT bonds. Ozone has a positive charge on one of its oxygen atoms, making it highly reactive and capable of accepting electrons from nucleophiles in a chemical reaction.

The method of the present application demonstrated the so-called “ozonative epoxidation” process for partial oxidation (epoxidation) of ethylene to ethylene oxide (EO) at room temperature. The current conventional ethylene epoxidation reaction on Ag-based catalysts is limited by the lack of sustained electrophilic oxygen on the catalyst leading to low conversion of ethylene. In the method of the present application, ozone was utilized for the generation of electrophilic oxygen for insertion into the ethylene double bond of ethylene, resulting in epoxide production. An advantage of this process is boosting selective oxygen coverage on the surface of the catalyst which enables achieving high EO selectivity while maintaining high conversion.

### Effect of Metal Loading and Surface Area of the Catalyst Support

To study the effect of metal loading and surface area of the catalyst support, catalysts with different supports and metal loadings were prepared and the results are summarized in Table 4.

TABLE 4
Ethylene catalytic ozonation results at room temperature
for ethylene oxide production (after 150 min of reaction)

| | Ethylene | Ethylene | O₃ |
|---|---|---|---|
| | Conversion | oxide | Conversion |
| Catalyst | (%) | Yield (%) | (%) |
| Ag (20%)/ γ-Al₂O₃ | 97 | 59 | 100 |
| Ag (20%)/ α-Al₂O₃ | 63.5 | 23.8 | 61.6 |
| Ag (10%)/TiO₂ anatase | 98.6 | 62.5 | 99.8 |
| Ag (10%)/ γ-Al₂O₃ | 96.7 | 74.2 | 99.8 |
| Ag (10%)/TiO₂ rutile | 100 | 31.3 | 92 |
| Ag (10%)/ZnO | 85 | 55 | 86 |
| Ag (5%)/ γ-Al₂O₃ | 98.5 | 57 | 99.7 |
| Ag (5%)/CeO₂ | 100 | 49 | 100 |
| Ag (5%)/TiO₂ rutile | 100 | 41 | 99.5 |
| Ag (2.5%)/ γ-Al₂O₃ | 74 | 36.4 | 55 |
| Ag (1%)/ γ-Al₂O₃ | 62 | 38 | 32 |

Catalysts with silver loading of 1 and 2.5 wt. % showed activity for catalytic ozonation of ethylene at room temperature. The stability of the catalysts with lower silver loading, such as 1 and 2.5 wt. % may be improved by optimizing the preparation method of the catalyst (e.g. calcination temperature) and/or by addition of promotors and/or addition of second metals and can be routinely determined by one skilled in the art. Examples of suitable second metals include, but are not limited to Au, Ba, Pd, Sn, Mn, Cu and the like. For example, by increasing the calcination temperature of AgOₓ/γ-Al₂O₃ with the silver of 2.5 wt. % from 350° C. to 750° C., conversion may increase from 74% to 93%. Similarly, adding 2.5 wt. % of Mn to AgOₓ/γ-Al₂O₃ with the silver of 2.5 wt. % can increase the conversion from 74% to 93%. Stable activity for ethylene oxide production was obtained over catalysts with silver loading of higher than 5 wt. %, namely 10 wt %. Further increase of silver loading from 10 to 20 wt. % did not improve the activity and selectivity of the catalysts.

Catalysts supported on semiconductors such as TiO₂ rutile, ZnO and CeO₂ nanoparticles with low surface area (15 m²/g, 25 m²/g and 8 m²/g, respectively) showed high activity and selectivity in catalytic ozonation of ethylene to produce ethylene oxide (Table 4). Higher activity was shown with high surface area γ-Al₂O₃ (219 m²/g) and TiO₂ anatase (420 m²/g) supports compared to the supports with lower surface area.

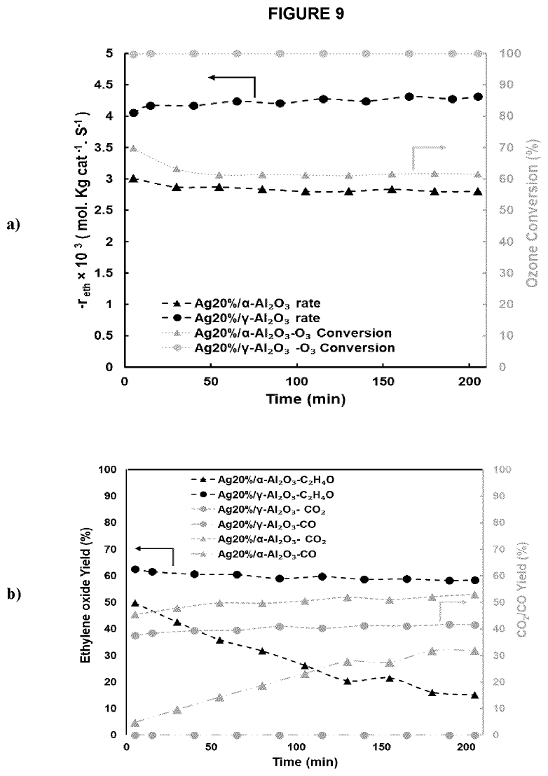

The results of the reaction rate and different product selectivity for the Ag catalysts loaded on the low surface area α-Al₂O₃ (surface area of 6 m²/g, very similar to the industrial catalyst) and high surface area γ-Al₂O₃ (219 m²/g) can be seen at FIG. 9 a-b. The Ag catalyst was loaded at 20 wt. %.

It can be seen in FIG. 9 a-b that low surface α-Al₂O₃ is less selective for ethylene oxide production in the presence of ozone.

While not wishing to be limited by theory, these results may be attributed to the fact that high surface area is optimal for ozone decomposition (more active site) specifically at room temperature. Ag/γ-Al₂O₃ showed higher activity in decomposing ozone (100% conversion) because of its higher surface area which could provide more electrophilic oxygen sites to selectively react with ethylene.

Furthermore, Ag/γ-Al₂O₃ showed higher selectivity to ethylene oxide compared to low surface area Ag/α-Al₂O₃. In addition, no CO was observed over Ag/γ-Al₂O₃ while CO concentration increased over time in presence of Ag/α-Al₂O₃. Thus, ethylene oxide production reaction at room temperature optimally includes a catalyst with high surface area. While not wishing to be limited by theory, this could be attributed to the presence of ozone. Ozone is adsorbed on the active sites of the catalyst and decomposes to produce active oxygen species. Therefore, these active species react with ethylene (mechanism could differ, ethylene might be adsorbed on the catalysts surface or be present in the gas phase). All these steps occur on the surface of the catalyst. In industrial ethylene oxide production, the low surface area is being used to decrease the contact time of the reactants on the catalyst surface and consequently decrease the secondary reactions such as total oxidation. But in the case of the present catalytic ozonation process, ozone decomposition may optimally use a high surface area. Also, the reaction of the decomposed ozone species and ethylene proceed through selective ethylene oxide production on the catalyst with a high surface area. While not wishing to be limited by theory, this may be because the high surface area of the catalysts overcomes the activation energy barrier of the reaction at room temperature.

### Effect of Support Type on Selectivity and Activity of the Catalyst

The type of the support and its interaction with Ag affects the activity and selectivity of the catalyst in ethylene oxide catalytic ozonation. Ag supported on semiconductors such as TiO₂, CeO₂, and ZnO was mainly in metallic form while supported on Al₂O₃ and zeolite supports Ag¹⁺ was dominant. Also, the type of the support considerably affected the selectivity of the Ag-based catalysts. Zeolite supports favored total oxidation of ethylene, while semiconductor supports produced ethylene oxide. Complete oxidation of ethylene to CO₂ and HO was achieved using AgOₓ/Beta catalyst at room temperature. It was shown that Ag supported on zeolites (including Beta, γ, and ZSM-5) favors total oxidation of ethylene in catalytic ozonation of ethylene and were not suitable supports for ethylene oxide production.

The catalysts of Ag supported on semiconductors such as TiO₂, CeO₂, and ZnO showed high activity and selectivity in ethylene oxide production as described above. Ag on these supports was already in metallic form and structural changes were observed for Ag after the reaction. The selected catalysts were highly active and selective in ethylene oxide production.

The selectivity towards ethylene oxide formation depends on ethylene conversion. Selectivity increases with decreased ethylene conversion. Higher values for ethylene oxide yield are obtained by decreasing the ethylene conversion (for example by increasing ethylene concentration or decreasing contact time). Low surface area supports may also be used in the method of the present application by keeping the conversion at a lower level to further increase the ethylene oxide yield, separate unreacted ethylene and recycle it to reactor.

Addition of promotors and inhibitors might further improve the ethylene oxide yield.

### FULL CITATIONS FOR DOCUMENTS REFERRED TO IN THE APPLICATION

[1] T. Pu, H. Tian, M. E. Ford, S. Rangarajan, I. E. Wachs, Overview of Selective Oxidation of Ethylene to Ethylene Oxide by Ag Catalysts, ACS Catal. (2019) 10727-10750. https://doi.org/10.1021/acscatal.9b03443.
[2] R. B. Grant, R. M. Lambert, A single crystal study of the silver-catalysed selective oxidation and total oxidation of ethylene, J. Catal. 92 (1985) 364-375. https://doi.org/10.1016/0021-9517 (85) 90270-2.
[3] W.-K. Y. Xing-Gui Zhou, Optimization of the fixed-bed reactor for ethylene epoxidation, Chem. Eng. Process. Process Intensif. 44 (2005) 1098-1107.
[4] A. Aho, K. Eränen, L. J. Lemus-Yegres, B. Voss, A. Gabrielsson, T. Salmi, D. Y. Murzin, Ethylene epoxidation over supported silver catalysts—influence of catalyst pretreatment on conversion and selectivity, J. Chem. Technol. Biotechnol. 93 (2018) 1549-1557. https://doi.org/10.1002/jctb.5592.
[5] A. J. F. Van Hoof, E. A. R. Hermans, A. P. Van Bavel, H. Friedrich, E. J. M. Hensen, Structure Sensitivity of Silver-Catalyzed Ethylene Epoxidation, (2019). https://doi.org/10.1021/acscatal.9b02720.
[6] S. I. R. Ebsdat, Ethylene oxide, Eur. Chem. News. 78 (2003) 13. https://doi.org/10.1002/14356007.a10.
[7] K. Fujiwara, K. Okuyama, S. E. Pratsinis, Metal-support interactions in catalysts for environmental remediation, Environ. Sci. Nano. 4 (2017) 2076-2092. https://doi.org/10.1039/c7en00678k.
[8] C. J. Pan, M. C. Tsai, W. N. Su, J. Rick, N. G. Akalework, A. K. Agegnehu, S. Y. Cheng, B. J. Hwang, Tuning/exploiting Strong Metal-Support Interaction (SMSI) in Heterogeneous Catalysis, J. Taiwan Inst. Chem. Eng. 74 (2017) 154-186. https://doi.org/10.1016/j.jtice.2017.02.012.
[9] A. R. Puigdollers, P. Schlexer, S. Tosoni, G. Pacchioni, Increasing oxide reducibility: The role of metal/oxide interfaces in the formation of oxygen vacancies, ACS Catal. 7 (2017) 6493-6513. https://doi.org/10.1021/acscatal.7b01913.
[10] M. Ahmadi, J. Timoshenko, F. Behafarid, B. R. Cuenya, Tuning the Structure of Pt Nanoparticles through Support Interactions: An in Situ Polarized X-ray Absorption Study Coupled with Atomistic Simulations, J. Phys. Chem. C. 123 (2019) 10666-10676. https://doi.org/10.1021/acs.jpcc.9b00945.
[11] S. T. OYAMA, Chemical and Catalytic Properties of Ozone, Catal. Rev. 42 (2000) 279-322. https://doi.org/10.1081/CR-100100263.
[12] M. Aghbolaghy, M. Ghavami, J. Soltan, N. Chen, Effect of active metal loading on catalyst structure and performance in room temperature oxidation of acetone by ozone, J. Ind. Eng. Chem. 77 (2019) 118-127. https://doi.org/10.1016/j.jiec.2019.04.026.
[13] H. Einaga, S. Futamura, ALUMINA-SUPPORTED METAL OXIDES FOR OXIDATION OF, 81 (2004) 121-128.
[14] A. J. F. van Hoof, E. A. R. Hermans, A. P. van Bavel, H. Friedrich, and E. J. M. Hensen, ACS Catal. 2019, 9, 9829-9839.
[15] Tiancheng Pu, Huijie Tian, Michael E. Ford, Srinivas Rangarajan, and Israel E. Wachs, ACS Catal. 2019, 9, 10727-10750.
[16] J. Ma, X. Li, C. Zhang, Q. Ma, H. He, Novel CeMnaOx catalyst for highly efficient catalytic decomposition of ozone, Appl. Catal. B Environ. 264 (2020). https://doi.org/10.1016/j.apcatb.2019.118498.
[17 ] M. Newville, Fundamentals of XAFS, Rev. Mineral. Geochemistry. 78 (2014) 33-74. https://doi.org/10.2138/rmg.2014.78.2.
[18] Rebsdat, S., Mayer, D., Ethylene oxide Ullmann's Encyclopedia Industrial Chemistry, Wiley-VCH Verlag Gmbh & Co. KGaA, Weinheim, (2001) 547-57.

1. A method for preparing an alkylene oxide from an alkene comprising:
reacting the alkene with ozone in the presence of a silver catalyst under conditions for selective partial oxidation of the alkene to provide the alkylene oxide, wherein the conditions comprise a temperature of about 0° C. to about 500° C.

2. The method of claim 1, wherein the temperature is about 25° C.

3. The method of claim 1, wherein the alkene is selected from ethylene, propylene, 1-butene, 2-butene, isobutylene, butadiene, 1-pentene and 2-pentene.

4. The method of claim 1, wherein the alkene is ethylene and the alkylene oxide is ethylene oxide.

5. The method of claim 1, wherein the silver catalyst is silver oxide.

6. The method of claim 1, wherein the silver catalyst is on a support, and wherein the support is Al₂O₃ or a semi-conducting metal oxide.

7. (canceled)

8. The method of claim 6, wherein the support is γ-Al₂O₃, α-Al₂O₃, TiO₂, ZnO or CeO₂.

9. The method of claim 8, wherein the support is γ-Al₂O₃.

10. (canceled)

11. The method of claim 9, wherein the γ-Al₂O₃ has a surface area of about 50 m²/g to about 500 m²/g.

12. The method of claim 9, wherein the surface area is of about 200 m²/g to about 240 m²/g.

13. (canceled)

14. The method of claim 8, wherein the support is TiO₂ anatase crystalline form.

15. The method of claim 8, wherein the TiO₂ anatase has a surface area of about 5 m²/g to about 500 m²/g.

16. The method of claim 14, wherein the surface area of TiO₂ anatase is of about 50 m²/g to about 450 m²/g.

17. (canceled)

18. The method of claim 6, wherein the silver catalyst is loaded on the support in an amount of about 0.1 wt % to about 30 wt % based on the total weight of the catalyst.

19. (canceled)

20. The method of claim 1, wherein the silver catalyst is 10 wt % AgOₓ supported on γ-Al₂O₃.

21. (canceled)

22. The method of claim 1, wherein the silver catalyst is 10 wt % AgO supported on TiO₂ anatase.

23. (canceled)

24. The method of claim 1, wherein the conditions for selective partial oxidation of the alkene to provide the alkylene oxide further comprise a partial pressure of the ozone of about 10×10⁻⁶ atm to about 0.8 atm.

25. (canceled)

26. The method of claim 1, wherein the silver catalyst after the partial oxidation comprises metallic silver.

27. The method of claim 1, wherein the silver catalyst is free from a promoter.

28. The method of claim 1, wherein the concentration ratio of alkylene to ozone is from about 1:5 to about 1:15.

29. (canceled)
